(19) United States
(12) Patent Application Publication
(10) Pub. No.: US 2025/0152937 A1
(43) Pub. Date: May 15, 2025

(54) Circuit Module for Pelvic Floor Muscle Rehabilitation Device and Pelvic Floor Muscle Rehabilitation Device

(71) Applicant: Shulin Chen, Shenzhen (CN)

(72) Inventor: Shulin Chen, Shenzhen (CN)

(21) Appl. No.: 19/025,992

(22) Filed: Jan. 16, 2025

(51) Int. Cl. A61N 1/36; A61L 2/10; A61N 1/05; A61N 1/372; A61N 1/378

(52) U.S. Cl. CPC A61N 1/36007; A61N 1/0524; A61N 1/36031; A61N 1/37247; A61N 1/3787; A61L 2/10; A61L 2202/24

(57) ABSTRACT

The present invention provides a circuit module for pelvic floor muscle rehabilitation device and pelvic floor muscle rehabilitation device. The circuit module includes a first controller, a wireless communication module, an electromyographic signal collection module and a discharge module. The pelvic floor muscle rehabilitation device includes the circuit module. The pelvic floor muscle rehabilitation device is connected to a terminal device through the wireless communication module, and sends command signals to the first controller. Users can check the electromyographic signal collection results and control the intensity of electrical stimulation through the terminal device.

### TECHNICAL FIELD

The present invention belongs to the technical field of pelvic floor muscle rehabilitation, and particularly relates to a circuit module for pelvic floor muscle rehabilitation device and pelvic floor muscle rehabilitation device.

### TECHNICAL BACKGROUND

Electromyographicity biofeedback training and neuromuscular electrical stimulation are physical therapies and are treatment methods for pelvic floor dysfunction. During the treatment, electrode slices need to be placed in the patient's vagina to collect the patient's electromyographic signals of the pelvic floor muscle surface or to emit pulsed electrical stimulation to achieve the treatment function.

In order to facilitate treatment, handheld pelvic floor muscle rehabilitation devices have appeared in the existing technology. They are equipped with built-in batteries and are portable and suitable for home use. However, this type of handheld pelvic floor muscle rehabilitation device only has the biofeedback function. The intensity of the electrical stimulation emitted by the electrode slices is fixed and cannot be adjusted. As a result, patients need to add additional physical rehabilitation training to achieve the effect of pelvic floor muscle rehabilitation treatment, and the patient's experience needs to be improved.

### INVENTION CONTENT

In order to overcome the defects of the existing technology, the present invention provides a circuit module for pelvic floor muscle rehabilitation device and pelvic floor muscle rehabilitation device. The pelvic floor muscle rehabilitation device includes the circuit module. The pelvic floor muscle rehabilitation device is connected to a terminal device through the wireless communication module, and sends command signals to the first controller. Users can check the electromyographic signal collection results and control the intensity of electrical stimulation through the terminal device.

The technical scheme adopted by the present invention for solving the technical problems is as follows:

A circuit module for pelvic floor muscle rehabilitation device includes a first controller, a wireless communication module, an electromyographic signal collection module, a discharge module and a battery. The first controller is electrically connected to the electromyographic signal collection module, the wireless communication module and the discharge module. The battery is electrically connected to the first controller for supplying power. The electromyographic signal collection module is used to collect electromyographic signals of the users and send the signals to the first controller. The discharge module is used to send electrical stimulation to users. The first controller can be connected to a terminal device through the wireless communication module.

Preferably, the circuit module also includes a wireless charging receiving module, which is electrically connected to the battery for recharging.

Preferably, the circuit module also includes a voltage module. The voltage module is electrically connected to the first controller and the battery, and is used to transform the battery voltage before supplying to the first controller.

Preferably, the circuit module also includes a high voltage relay module. The electromyographic signal collection module is electrically connected to the first controller and the high voltage relay module. And the electromyographic signal collection module is used to collect electromyographic signals of users and then send the signals to the first controller; the discharge module is electrically connected to the first controller and the high voltage relay module, and is used to send electrical stimulation to users. The first controller is used to turn on or off the high voltage relay module. When the high voltage relay module turn on, the electromyographic signal collection module is connected to the first controller; when the high voltage relay module turn off, the discharge module is connected to the first controller.

Preferably, the circuit module also includes a motor driving module, which is electrically connected to the first controller.

The present invention also provides a pelvic floor muscle rehabilitation device, applying the circuit module as described above. The pelvic floor muscle rehabilitation device also includes a rehabilitation device body, which includes a rehabilitation working area and a holding area connected to the rehabilitation working area. The surface of the rehabilitation working area is provided with electrode slices, which are used to send electrical stimulation to users and collect the electromyographic signals of users. The rehabilitation device body is provided with an inner cavity provided with a battery and a first control mainboard inside. The battery is electrically connected to the first control mainboard, and the circuit module is arranged on the first control mainboard.

Preferably, a connection board is arranged in the inner cavity. The first controller is arranged inside the holding area. The connection board is arranged inside the rehabilitation working area and two ends of the connection board are respectively connected to the electrode slices and the first controller.

Preferably, the rehabilitation device body also includes a charging case, which includes a bottom holder and an upper cover for covering the bottom holder. The bottom holder is provided with a trough adapted to the rehabilitation device body. A second controller and a wireless charging sending module are arranged inside the bottom holder.

Preferably, an ultraviolet light driving module and an ultraviolet light module are arranged inside the bottom holder. The ultraviolet light driving module is electrically connected to the second controller and the ultraviolet light module. The ultraviolet light is arranged in the ultraviolet light module and embedded in the inner wall of the trough.

Preferably, a Hall detection module is arranged inside the bottom holder. The Hall detection module is electrically connected to the second controller. A first magnet is arranged beneath the upper cover.

The beneficial effects of the present invention:

The present invention provides a circuit module for pelvic floor muscle rehabilitation device and pelvic floor muscle rehabilitation device. The circuit module includes a first controller, a wireless communication module, an electromyographic signal collection module and a discharge module. The pelvic floor muscle rehabilitation device includes the said circuit module. The pelvic floor muscle rehabilitation device can be connected to a terminal device through the wireless communication module, and sends command signals to the first controller. Users can check the electromyographic signal collection results and control the intensity of electrical stimulation through the terminal device.

### DESCRIPTION OF FIGURES

FIG. 1 is a functional block diagram of the Embodiment I of the present invention;

FIG. 2 is a circuit structure diagram of a first controller in the Embodiment I of the present invention;

FIG. 3 is a circuit structure diagram of a wireless charging receiving module in the Embodiment I of the present invention;

FIG. 4 is a circuit structure diagram of a touch module in the Embodiment I of the present invention;

FIG. 5 is a circuit structure diagram of a boost circuit in a voltage module in the Embodiment I of the present invention;

FIG. 6 is a circuit structure diagram of a buck circuit in a circuit module in the Embodiment I of the present invention;

FIG. 7 is a circuit structure diagram of the electromyographic signal collection module in the Embodiment I of the present invention;

FIG. 8 is a circuit structure diagram of a boost module in the Embodiment I of the present invention;

FIG. 9 is a circuit structure diagram of a discharge module in the embodiment I of the present invention;

FIG. 10 is a circuit structure diagram of a wireless communication module in the Embodiment I of the present invention;

FIG. 11 is a circuit structure diagram of a high voltage relay module in the Embodiment I of the present invention;

FIG. 12 is a circuit structure diagram of a motor driving module in the Embodiment I of the present invention;

FIG. 13 is a circuit structure diagram of a high voltage detection module in the Embodiment I of the present invention;

FIG. 14 is a circuit structure diagram of a second controller in the Embodiment II of the present invention;

FIG. 15 is a circuit structure diagram of a wireless charging sending module in the Embodiment II of the present invention;

FIG. 16 is a circuit structure diagram of a Hall detection module in the Embodiment II of the present invention;

FIG. 17 is a circuit structure diagram of a three-color light module in the Embodiment II of the present invention;

FIG. 18 is a circuit structure diagram of an ultraviolet light driving module in the Embodiment II of the present invention;

FIG. 19 is a circuit structure diagram of an ultraviolet light in the Embodiment II of the present invention;

FIG. 20 is a schematic diagram of a charging case in the Embodiment II of the present invention;

FIG. 21 is a schematic diagram of the rehabilitation device body in the Embodiment II of the present invention;

FIG. 22 is a schematic diagram of the partial structure of the rehabilitation device body in the Embodiment II of the present invention;

FIG. 23 is an exploded view of the charging case in the Embodiment II of the present invention;

FIG. 24 is a schematic diagram of a second inner cover in the Embodiment II of the present invention;

### DESCRIPTION OF MARKS IN FIGURES

1-rehabilitation device body;

11-rehabilitation working area;

111-electrode slice;

1111-first electrode slice;

1112-second electrode slice;

112-connection board;

12-holding area;

121-first control mainboard;

122-battery;

123-motor;

124-touch button;

125-spring;

126-wireless charging receiver;

2-charging case;

21-bottom holder;

211-trough;

2111-ultraviolet light;

212-second control mainboard;

213-first outer case;

214-first inner case;

215-first limited column;

216-second magnet;

22-upper cover;

221-first magnet;

222-second outer cover;

223-second inner cover;

224-second limited column;

225-third magnet;

23-charging port.

### DETAILED DESCRIPTION OF THE EMBODIMENTS

Below is a further detailed description of the present invention based on the figures.

The present embodiment only shows an explanation of the present invention and it is not a limitation to the present invention. The skilled in the art can make modifications to this embodiment as needed without making any creative contributions after reading this specification, which are always protected by the patent law as long as they are within the scope of the claims of the present invention.

It should be noted that when an element is called as being “fixed to” or “arranged on” another element, it can be directly on the other element or indirectly on the other element. When an element is called as being “connected to” another element, it can be directly connected to the other element or indirectly connected to the other element.

It should be noticed that the terms “length”, “width”, “above”, “below”, “front”, “rear”, “left”, “right”, “vertical”, “horizontal”, “top”, “bottom”, “inside” and “outside” which indicates the orientations or positional relationships are based on the orientations or positional relationships shown in the figures. They are only for facilitating describing the present invention and simplifying the description, rather than indicating or implying that the device or component must have a specific orientation, construct and operate in a specific orientation, therefore, it understood as a limitation of the present invention.

In addition, the terms “first” and “second” are used for descriptive purposes only and cannot be understood as indicating or implying relative importance or implicitly indicating the quantity of indicated technical features. Therefore, a feature defined as “first” and “second” may explicitly or implicitly include one or more of these features. In the description of the present invention, “a plurality of” means two or more, unless otherwise specifically defined.

The present invention provides a circuit module for pelvic floor muscle rehabilitation device and a pelvic floor muscle rehabilitation device. The pelvic floor muscle rehabilitation device includes the circuit module, which includes a first controller, a wireless communication module, an electromyographic signal collection module and a discharge module. The pelvic floor muscle rehabilitation device is connected to a terminal device through the wireless communication module, and sends command signals to the first controller. Users can check the electromyographic signal collection results and control the intensity of electrical stimulation through the terminal device.

### Embodiment I

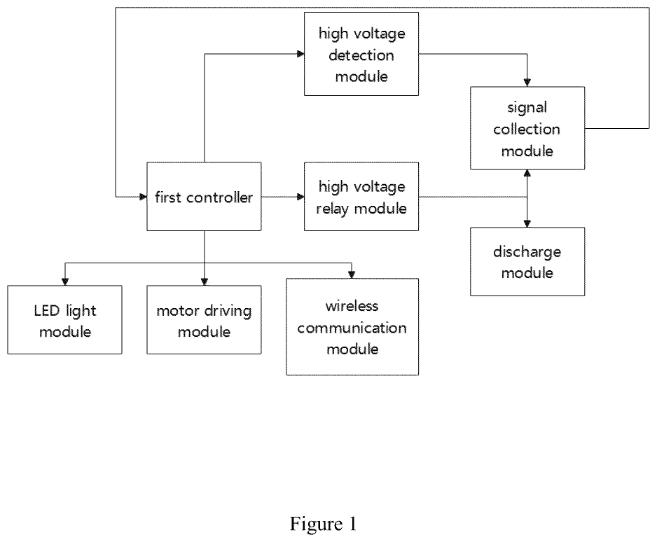

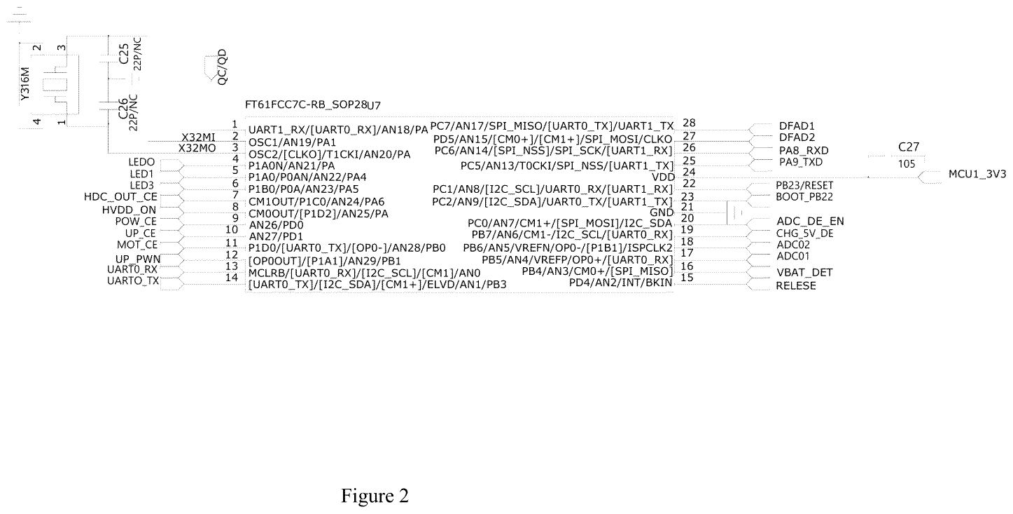

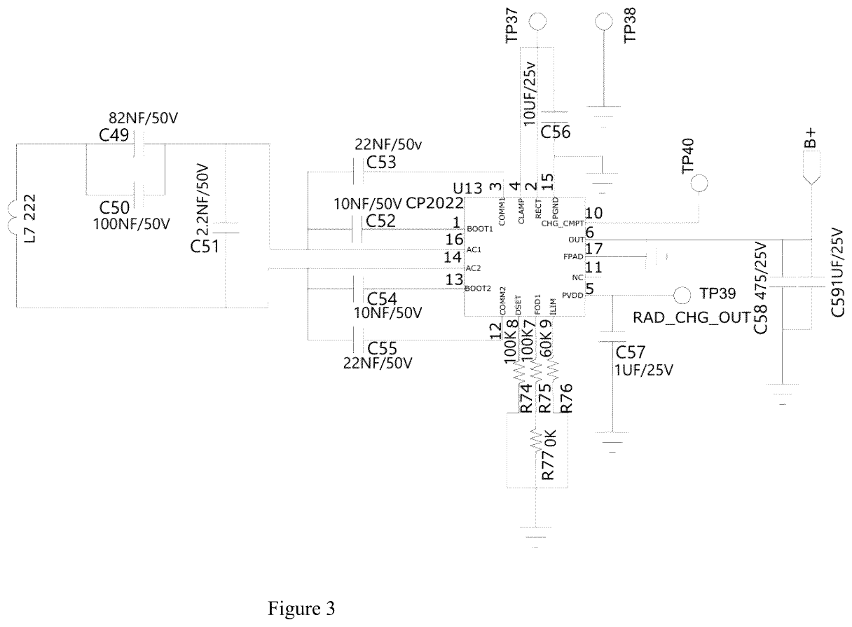

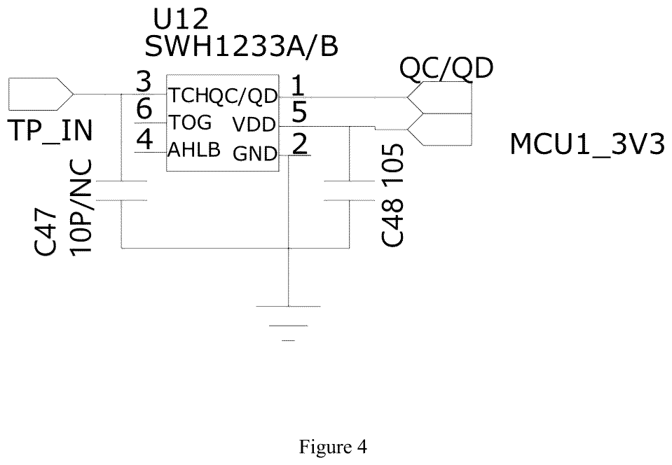

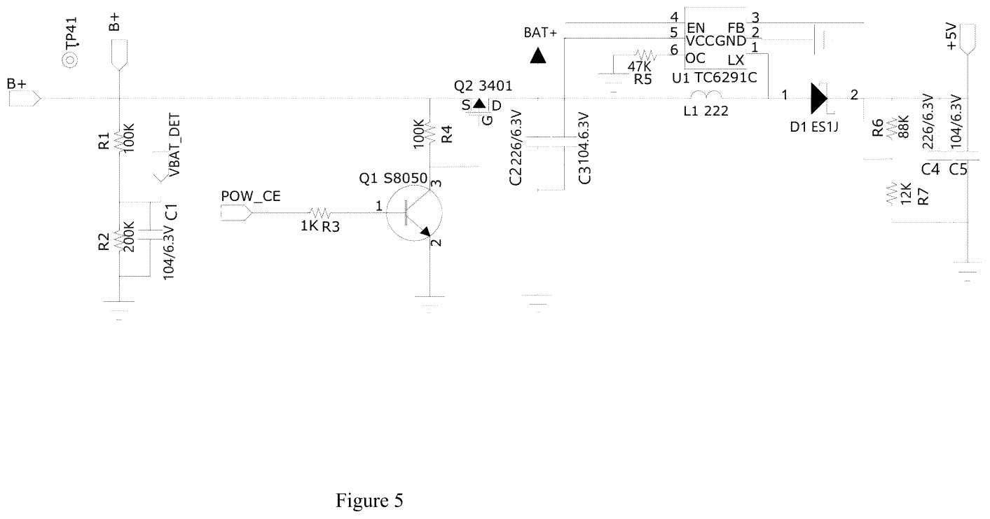

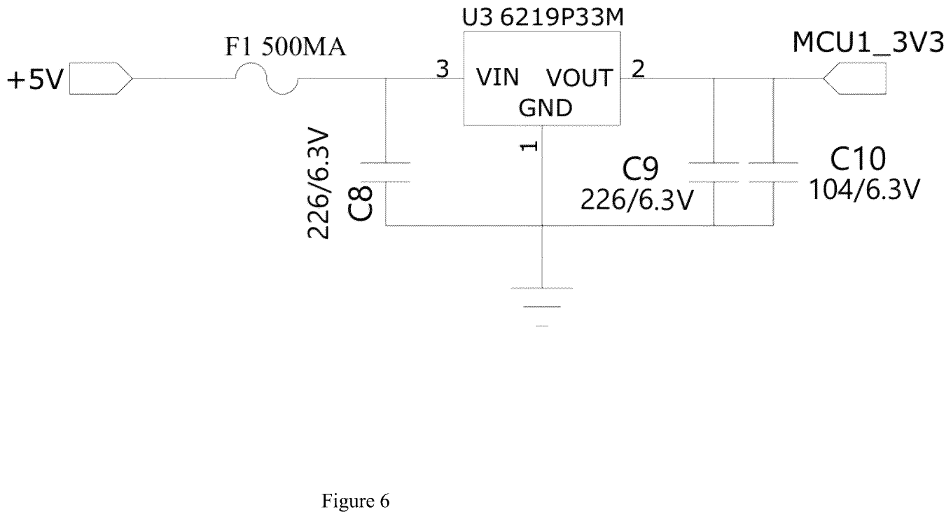

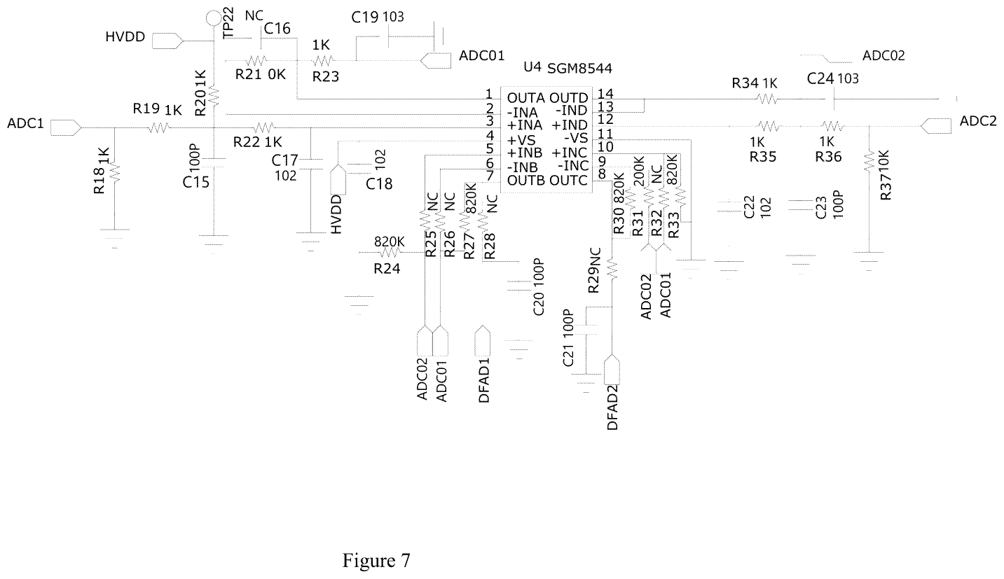

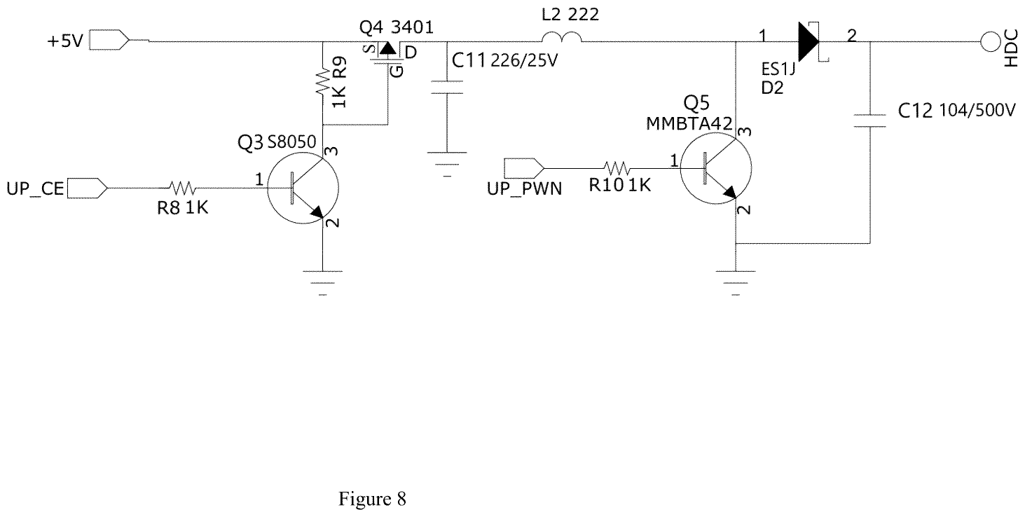

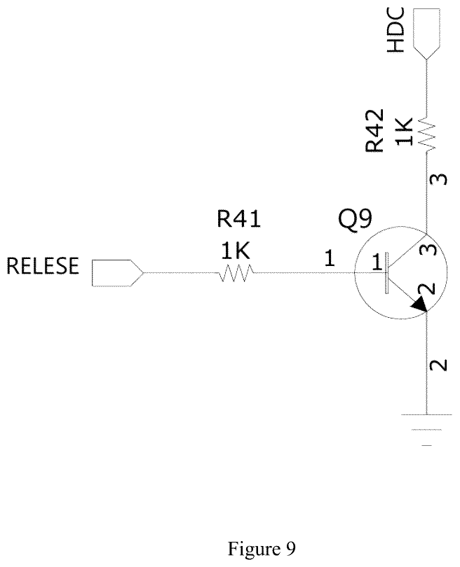

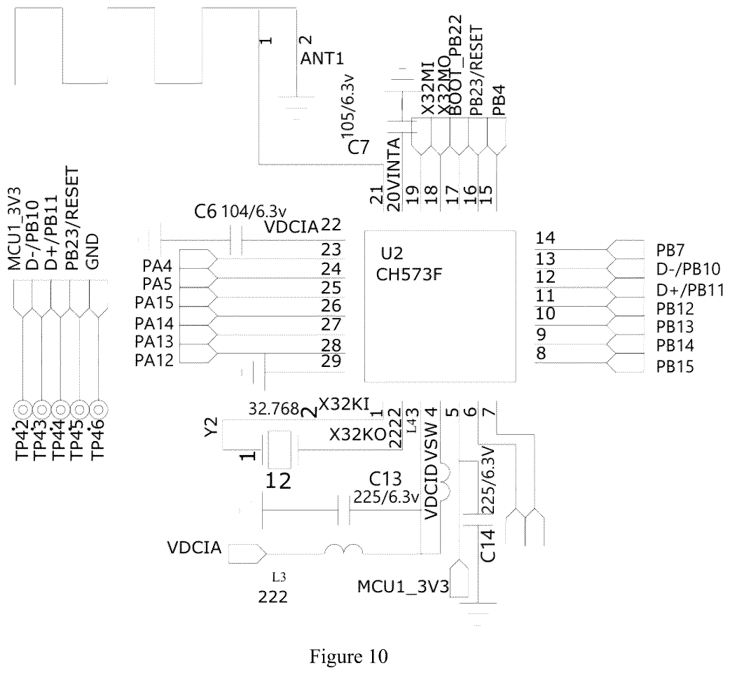

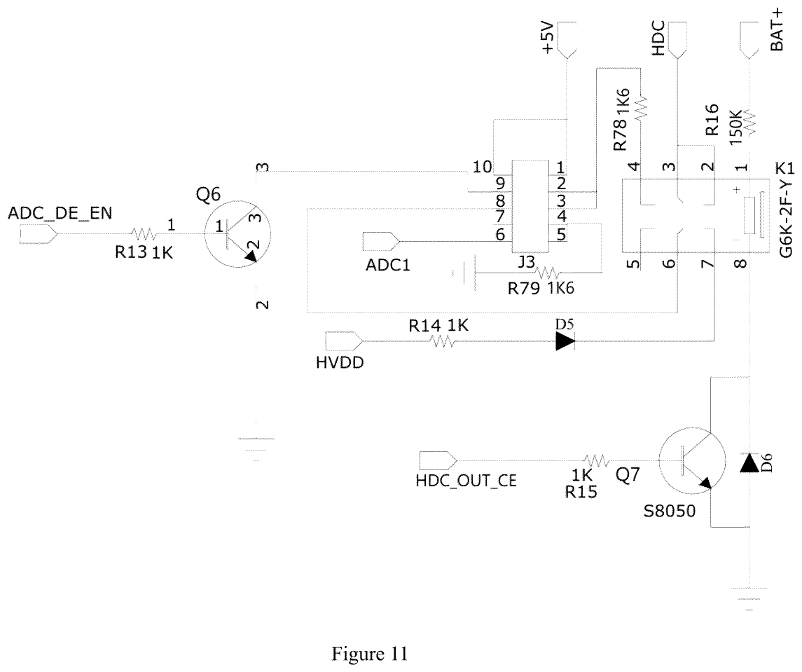

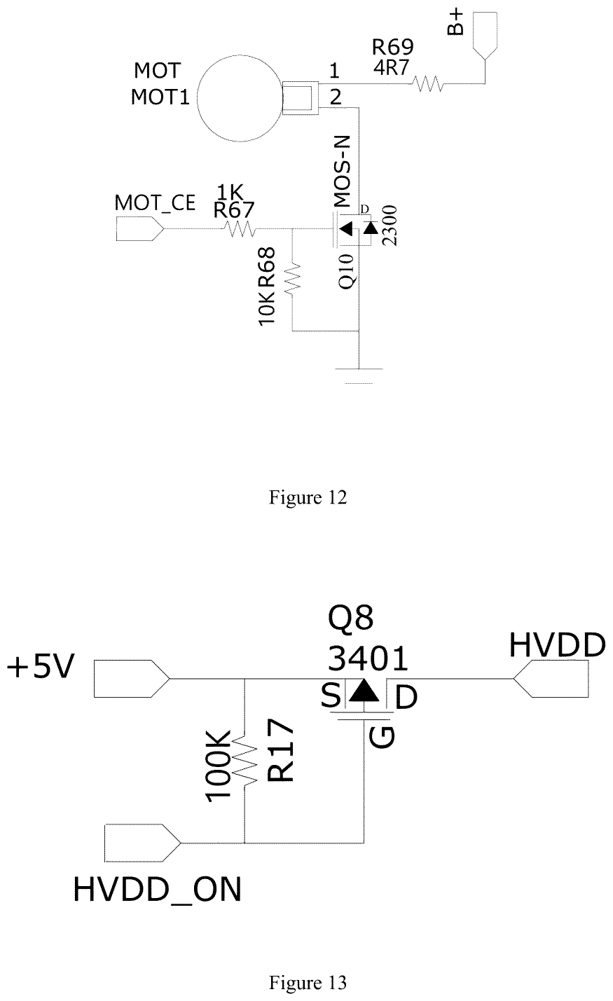

Referring to FIGS. 1 to 13, this embodiment provides a circuit module for pelvic floor muscle rehabilitation device. The circuit module including a first controller, a wireless charging receiving module, a voltage module, a boost module, an electromyographic signal collection module, a discharge module, a wireless communication module, a high voltage relay module, a motor driving module, a touch module and a high voltage detection module. The circuit module is connected to a battery, which is used as a power source to provide voltage for the circuit module. The wireless charging receiving module is electrically connected to the battery for recharging.

In this embodiment, the first controller uses a single chip microcomputer U7 of the model FT62FCC7C-RB-SOP28, hereinafter referred to as the first controller U7. The first controller U7 includes 28 pins. Pin 24 of the first controller U7 is connected to the voltage module, and pin 21 of the first controller U7 is grounded.

The voltage module is electrically connected to the first controller U7 and the battery, and is used to boost the voltage provided by the battery to a voltage of +5V. And step down the voltage to a voltage of +3.3V and then send the voltage of +3.3V to the first controller U7, thereby providing an operation voltage for the first controller U7.

The touch module is electrically connected to the first controller U7. The touch module is used to receive touch signals and send the touch signals to the first controller U7. After receiving the touch signals, the first controller U7 outputs a high level to enable the voltage module to generate an operation voltage.

Specifically, the touch module includes a chip U12. Pin 3 of the chip U12 is connected to a touch pin, and pin 1 of the chip U12 is connected to pin 1 of the first controller U7. When the touch pin is touched, a touch signal can be sent to the first controller U7 through the chip U12.

The voltage module includes a triode Q1, a field effect transistor Q2, a boost chip U1 and a buck chip U3.

Specifically, a collector of triode Q1 is connected to the battery, a base of triode Q1 is connected to pin 9 of the first controller U7, and an emitter of transistor Q1 is grounded; a resistor R4 is connected in series between triode Q1 and the battery. The node between the battery and resistor R4 is grounded after passing through resistors R1 and R2. Capacitor C1 is connected in parallel at two ends of resistor R2, and pin 16 of the first controller U7 is connected to capacitor C1. The source and gate of field effect transistor Q2 are connected in parallel at two ends of the resistor R4, and the drain of field effect transistor Q2 is connected to boost chip U1. When the first controller U7 receives the touch signal, it outputs a high level to turn on triode Q1 and field effect transistor Q2. The voltage output by the battery generates a +5V voltage after passing through boost chip U1, and the +5V voltage is connected to buck chip U3. Pin 1 of buck chip U3 is grounded, and pin 2 of buck chip U3 outputs a +3.3V voltage and is connected to pin 24 of the first controller U7.

The electromyographic signal collection module is electrically connected to the first controller U7 and the high voltage relay module, and is used to collect the electromyographic signals of users and then send the signals to the first controller U7; the discharge module is electrically connected to the first controller U7 and the high voltage relay module, and is used to send electrical stimulation to users. The high-voltage relay module acts as a converter, and the first controller U7 is used to turn on or off the high voltage relay module. when the high voltage relay module turn on, the electromyographic signal collection module is connected to the first controller; when the high voltage relay module turn off, the discharge module is connected to the first controller.

Specifically, the electromyographic signal collection module includes an operational amplifier U4. Pin 1 of the operational amplifier U4 is connected to pin 17 of the first controller U7, pin 14 of the operational amplifier U4 is connected to pin 18 of the first controller U7, pin 8 of the operational amplifier U4 is connected to pin 27 of the first controller U7, and pin 7 of the operational amplifier U4 is connected to pin 28 of the first controller U7. Pin 3 of the operational amplifier U4 is connected to an electrode slice, through which the electromyographic signals are collected. After sending the collected electromyographic signals to the operational amplifier U4, the electrode slice converts the collected electromyographic signals into digital acquisition signals, and then sends the digital acquisition signals to the first controller U7.

The boost module is electrically connected to the first controller U7, and is used to boost the +5V voltage output from the voltage module to the voltage required by the discharge module.

Specifically, the boost module includes a triode Q3, a field effect transistor Q4, a triode Q5 and an inductor L2. The base of the triode Q3 is connected to pin 10 of the first controller U7, the base of the triode Q5 is connected to pin 12 of the first controller U7. The collector of the triode Q3 is connected in series to one end of the resistor R9, and other end of the resistor R9 is connected to the +5V voltage output by the voltage module. The source and gate of the field effect transistor Q4 are connected in parallel at two ends of the resistor R9, the drain of the field effect transistor Q4 is connected to one end of the inductor L2. Other end of the inductor L2 has two connection points, one of which is connected to the collector of the triode Q5, and other connection point is connected to the high voltage relay module. The source of the triode Q5 is connected to pin 12 of the first controller U7, and the emitter of the triode Q5 is grounded.

When electrical stimulation is required, the first controller U7 outputs a control signal to control the boost voltage circuit composed of triode Q3, field effect transistor Q4, triode Q5 and inductor L2 to boost the +5V voltage output by the voltage module to the voltage required by the discharge module, and send the voltage to the high voltage relay module, and then to the discharge module.

The discharge module includes a triode Q9. The base of the triode Q9 is connected to the 15th pin of the first controller U7, the collector of the triode Q9 is connected to the high voltage relay module, and the emitter of the triode Q9 is grounded. When the first controller U7 outputs a high level to the triode Q9, the high voltage electricity generated by the boost module can be discharged to the earth wire terminal through the triode Q9 to achieve the discharge function, which can ensure that there is no voltage output when not in use, avoiding safety hazards.

The wireless communication module is electrically connected to the first controller U7, and can connect the first controller U7 and the terminal device. The terminal device referred to here can be a mobile phone or a tablet, etc. The first controller U7 can send the collected digital signals to the terminal device, and users can view relevant information or control the first controller U7 through the terminal device.

Specifically, the wireless communication module includes a Bluetooth chip U2. The pin 5 of the Bluetooth chip U2 is connected to the +3.3V voltage output by the voltage module, the pin 6 of the Bluetooth chip U2 is connected to the pin 26 of the first controller U7, and the pin 7 of the Bluetooth chip U2 is connected to the pin 25 of the first controller U7. Through the wireless communication module, the first controller U7 is connected with the external terminal device. The terminal device referred to here can be an electronic device such as a mobile phone or a tablet. Users can check the electromyographic signals collected by the electromyographic signal collection module through the terminal device, and can also send instructions to the first controller U7 through the terminal device to adjust the intensity of the discharge module.

The high voltage relay module includes a triode Q6, a triode Q7, a relay K1, a diode D6 and a connector J3.

Specifically, the base of triode Q6 is connected to pin 20 of the first controller U7, the emitter of triode Q6 is grounded, and the collector of triode Q6 is connected to relay K1 through connector J3. The anode of diode D6 is grounded, the cathode of diode D6 is connected to pin 8 of relay K1. The base of triode Q7 is connected to the first controller U7, the collector of triode Q7 is connected to the node between the cathode of diode D6 and relay K1, and the emitter of triode Q7 is connected to the node between the anode of diode D6 and the ground. Pin 1 of relay Kl is connected to the power source, pin 4 of relay K1 is connected to the electromyographic signal collection module through connector J3, and pin 3 of relay Kl is connected to the discharge module. By outputting a high level or a low level to triode Q7 by the first controller U7, the connection relationship of relay K1 can be switched, so that the electromyographic signal collection module is connected to the first controller U7, or the discharge module is connected to the first controller U7.

The motor driving module is electrically connected to the first controller U7, which is used to turn on or off the motor driving module.

Specifically, the motor driving module includes a field effect transistor Q10 and a motor MOT1. The source of the field effect transistor Q10 is connected to pin 11 of the first controller U7, the gate of the field effect transistor Q10 is grounded, the drain of the field effect transistor Q10 is connected to pin 2 of the motor MOT1. Pin 1 of the motor MOT1 is connected to the power source.

The high voltage detection module is electrically connected to the first controller U7 and the electromyographic signal collection module, and is used to provide a reference voltage for the electromyographic signal collection module.

Specifically, the high-voltage detection module includes a field effect transistor Q8, the source of the field effect transistor Q8 is connected to the +5V voltage output by the boost module, the gate of the field effect transistor Q8 is connected to the pin 8 of the first controller U7, and the drain of the field effect transistor Q8 is connected to the electromyographic signal collection module. When the first controller U7 outputs a high level to the field effect transistor Q8, the field effect transistor Q8 is turned on to provide a reference voltage for the electromyographic signal collection module.

### Embodiment II

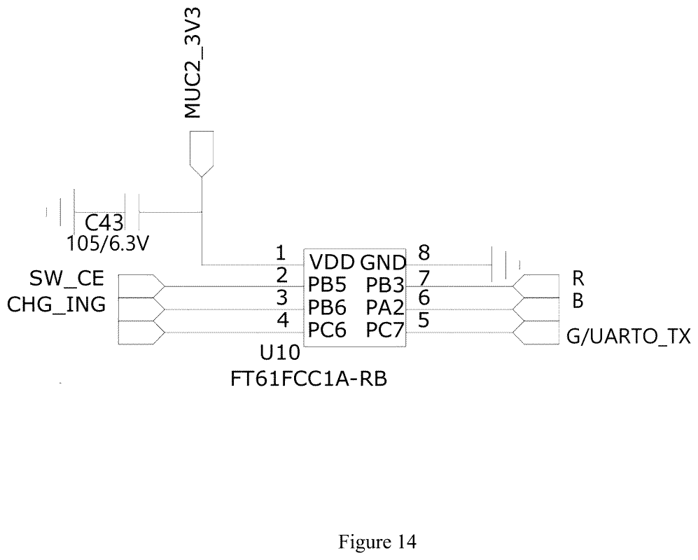

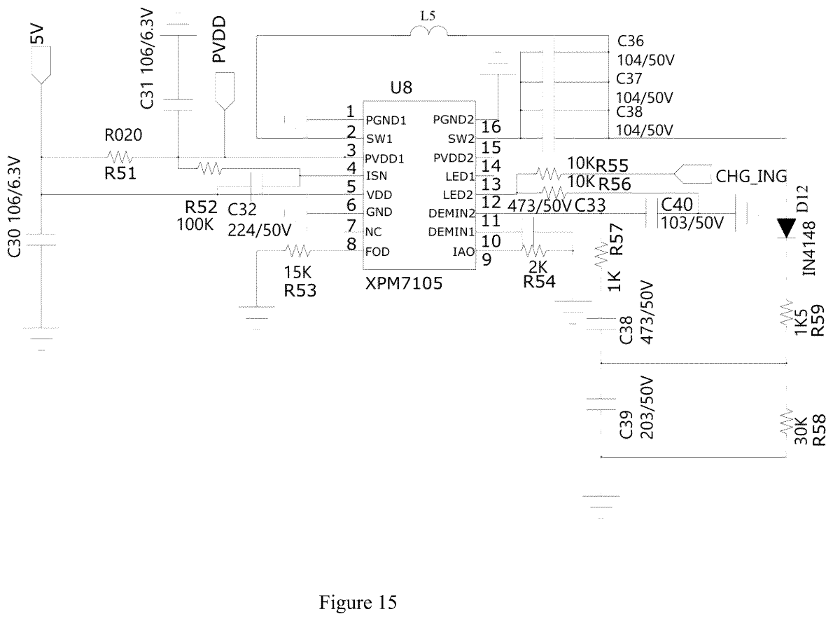

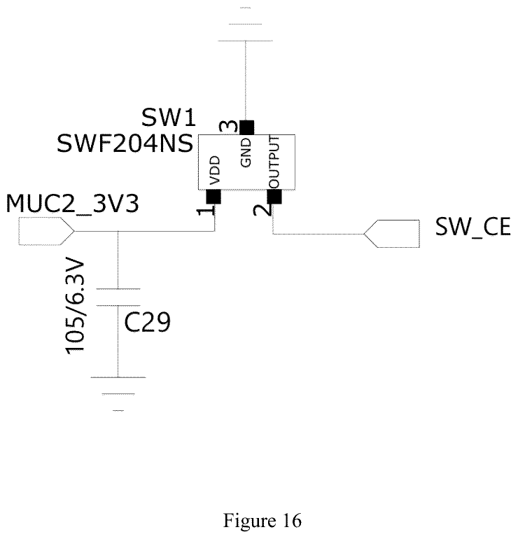

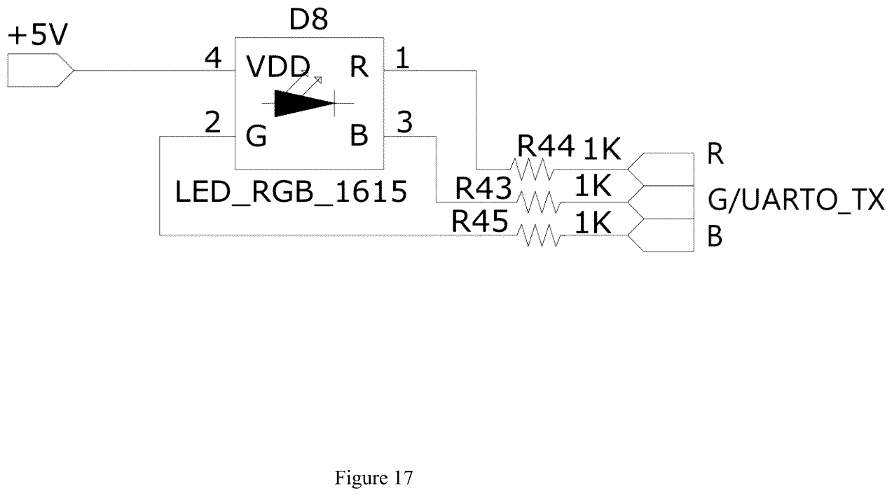

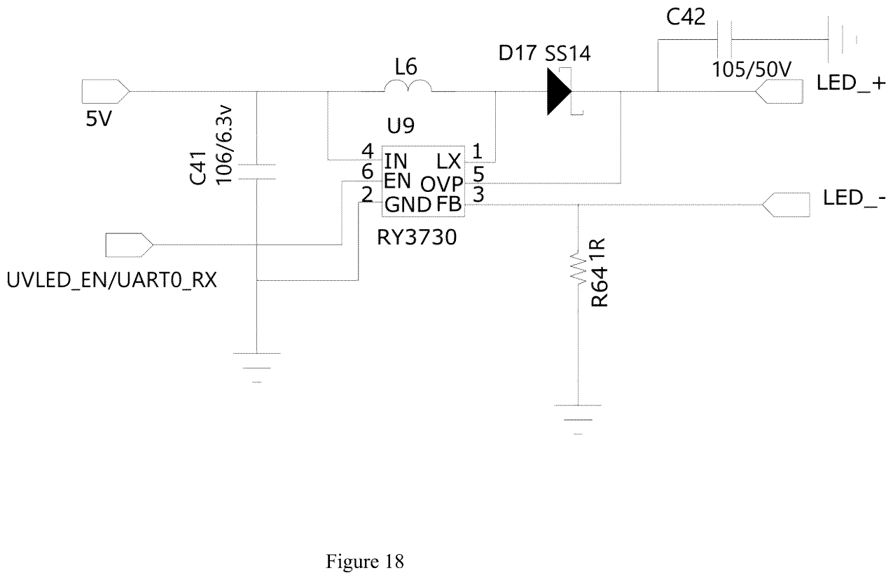

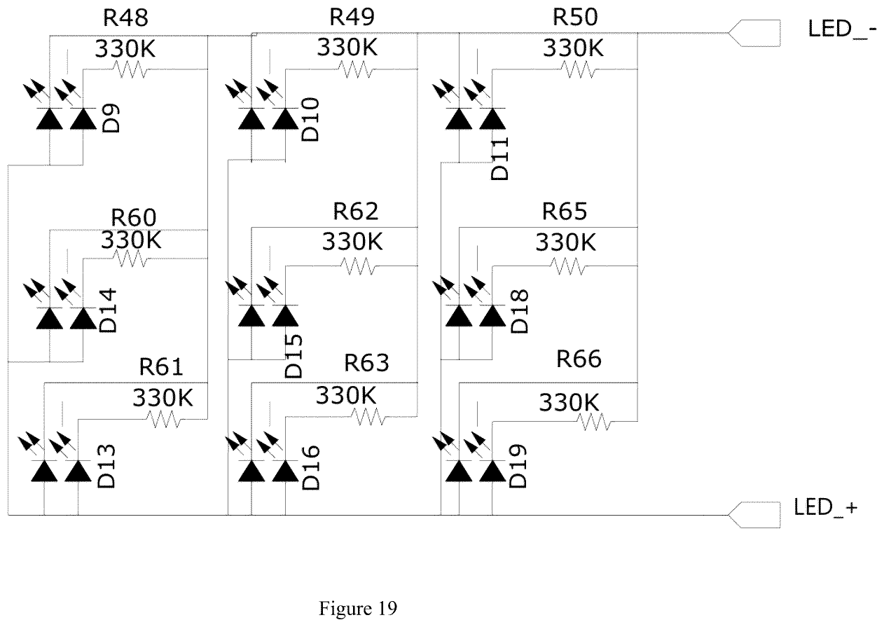

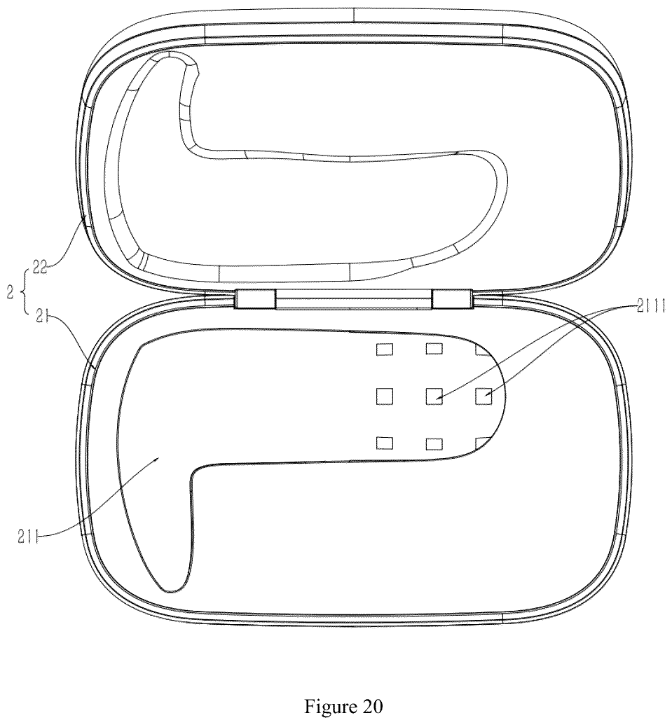

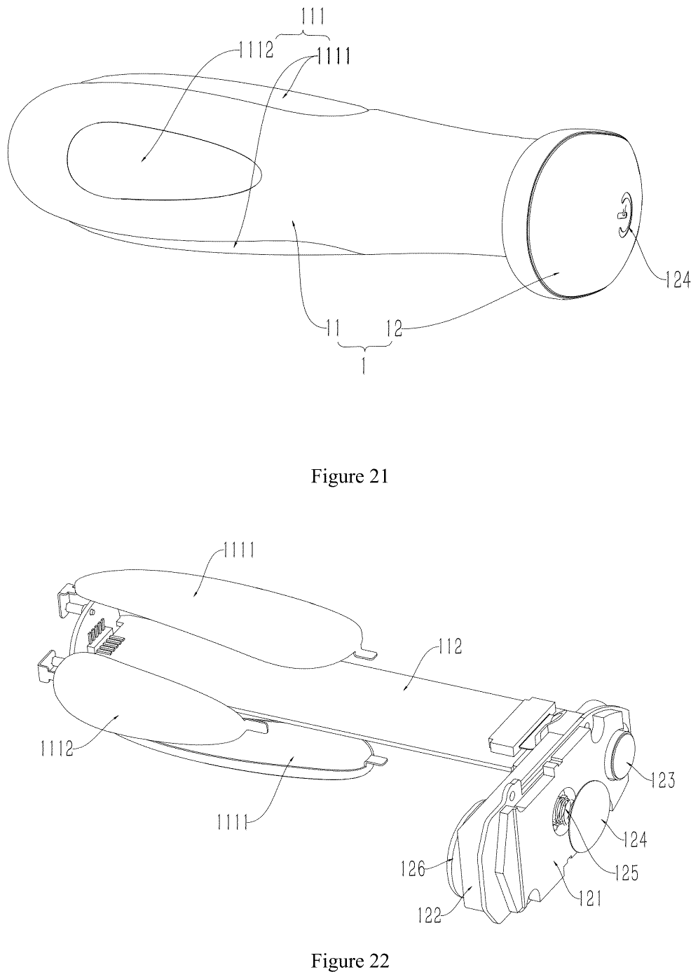

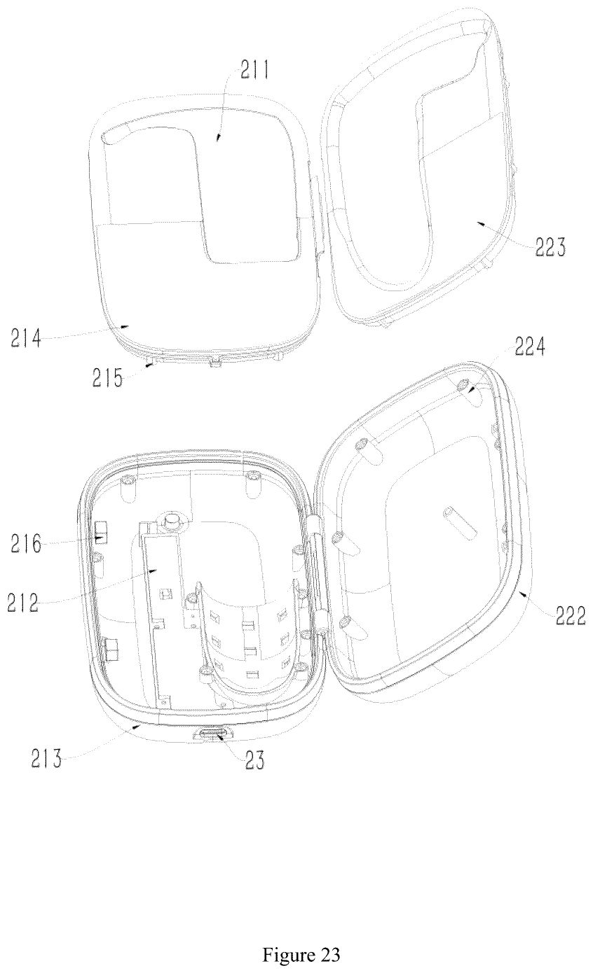

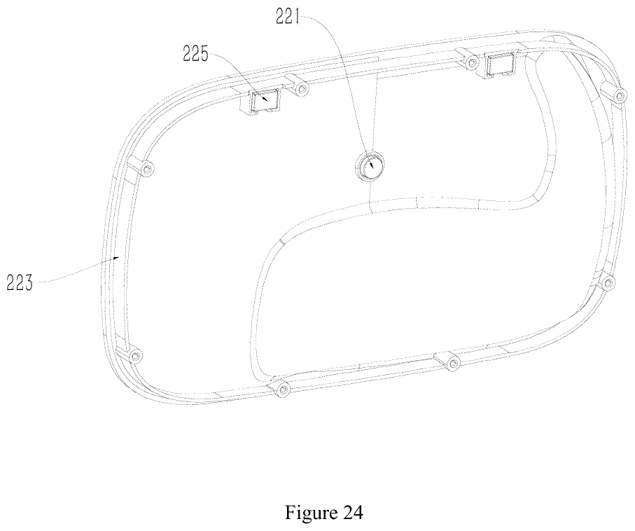

Referring to FIGS. 14 to 24, this embodiment provides a pelvic floor muscle rehabilitation device, including the circuit module in the Embodiment I.

Specifically, the pelvic floor muscle rehabilitation device includes a rehabilitation device body 1, which includes a rehabilitation working area 11 and a holding area 12 connected to the rehabilitation working area 11. The surface of the rehabilitation working area 11 is provided with electrode slices 111, which are used to send electrical stimulation to users and collect the electromyographic signals of users. The rehabilitation device body 1 is provided with an inner cavity provided with a battery 122 and a first control mainboard 121 inside. The battery 122 is electrically connected to the first control mainboard 121, and the circuit module is arranged on the first control mainboard 121.

The electrode slices 111 includes first electrode slices 1111 and a second electrode slice 1112. In this embodiment, two first electrode slices 1111 are arranged, and one second electrode slice 1112 is arranged. The second electrode slice 1112 is arranged between the two first electrode slices 1111. The first electrode slices 1111 are electrically connected to the discharge module, and the discharge module sends current to the first electrode slices 1111 to electrically stimulate the electromyographic system of the pelvic floor muscle; the second electrode slice 1112 is electrically connected to the electromyographic signal collection module, and the second electrode slice 1112 collects the electromyographic signals of users and sends the collected electromyographic signals to the first controller U7. Specifically, the first electrode slices 1111 are stimulation electrode slices, and the second electrode slice 1112 is an electromyography electrode slice.

A touch pin is arranged on the first control mainboard 121, a touch button 124 is arranged on the surface of the holding area 12. A spring 125 is arranged at the bottom of the touch button 124, and the bottom of the spring 125 corresponds to the touch pin on the first control mainboard 121. After the touch button 124 is pressed, the spring 125 can provide elastic reset force for the touch button 124. When users touch the touch button 124, the touch pin is triggered, and the touch module sends the touch signal to the first controller U7, which responds to the touch signal.

Preferably, a connection board 112 is arranged in the inner cavity. The first control mainboard 121 is arranged inside the holding area 12. The connection board 112 is arranged inside the rehabilitation working area 11. Two ends of the connection board 112 are respectively connected to the electrode slices 111 and the first controller U7 to send signal.

Preferably, a motor 123 is also arranged in the rehabilitation device body 1. The motor 123 is arranged inside the holding area 12. The motor 123 is electrically connected to the first controller U7 on the first control mainboard 121 through the motor 123 driving module. The first controller U7 controls the opening and closing of the motor 123. When the rehabilitation device in this embodiment is successfully connected to the terminal device through the wireless communication module, the first controller U7 controls the motor 123 to turn on, and the rehabilitation device body 1 vibrates.

Preferably, a wireless charging receiver 126 is also arranged inside the rehabilitation device body 1, and the wireless charging receiver 126 is electrically connected to the first control mainboard 121. Further, the pelvic floor muscle rehabilitation device of this embodiment also includes a charging case 2, which includes a bottom holder 21 and an upper cover 22 for covering the bottom holder 21. The bottom holder 21 is provided with a trough 211 adapted to the rehabilitation device body 1. A second control mainboard 212 is arranged inside the bottom holder 21.

The second control mainboard 212 is provided with a wireless charging sending module and a second controller. The wireless charging sending module is electrically connected to the second controller.

In this embodiment, the second controller uses a chip U10 of model FT61FCC1A-RB, hereinafter referred to as the second controller U10. The wireless charging sending module is electrically connected to pin 3 of the second controller U10 and can feed back the charging status to the second controller U10.

Further, the charging case 2 is provided with a charging port 23, which is electrically connected to the wireless charging sending module. The charging port 23 is connected to an external power source. When the user places the rehabilitation device body 1 in the trough 211 on the bottom holder 21 of the charging case 2, the wireless charging sending module in the charging case 2 will cooperate with the wireless charging receiving module in the rehabilitation device body 1 to supply power for the battery 122 in the rehabilitation device body 1.

Preferably, an ultraviolet light driving module and an ultraviolet light module are arranged inside the bottom holder 21. The ultraviolet light driving module is electrically connected to the pin 4 of the second controller U10. The ultraviolet light 2111 is arranged in the ultraviolet light module and is embedded in the side wall of the trough 211. The ultraviolet light driving module is electrically connected to the ultraviolet light module. The second controller U10 controls the ultraviolet light driving module to control the ultraviolet light 2111 in the ultraviolet light module to turn on or off. When the rehabilitation device is placed in the trough 211 and the ultraviolet light 2111 is turned on, the rehabilitation device body 1 can be disinfected.

Further, a Hall detection module is arranged inside the bottom holder 21, which is electrically connected to the second controller U10. A first magnet 221 is arranged beneath the upper cover 22. Specifically, the Hall detection circuit includes a Hall sensor SW1, whose pin 1 is connected to a power source. An intermediate node between the power source and pin 1 of the Hall sensor SW1 is grounded after passing through a capacitor C29. Pin 2 of the Hall sensor SW1 is connected to pin 2 of the second controller U10, and pin 3 of the Hall sensor SW1 is grounded.

When the upper cover 22 is covered on the bottom holder 21, the first magnet 221 triggers the Hall sensor SW1 in the Hall detection module, and the Hall sensor SW1 sends the trigger signal to the second controller U10. When the second controller U10 receives the trigger signal, it controls the ultraviolet light 2111 in the ultraviolet light module and the wireless charging sending module to turn on. In other words, when the user places the rehabilitation device body 1 in the trough 211 of the battery 122 and covers the charging case 2 on the bottom holder 21, the ultraviolet light 2111 will automatically turn on to disinfect the rehabilitation device body 1. At the same time, the rehabilitation device body 1 enters the wireless recharging state.

Further, the second control mainboard 212 is provided with a three-color light module, which is electrically connected to the second controller U10. Specifically, the three-color light module includes an RGB LED light bead. The pin 4 of the RGB LED light bead is connected to the power source, and the pin 3 of the RGB LED light bead is electrically connected to the pin 6 of the second controller U10. The second controller U10 can control the three-color light to emit different colors of light according to the current working condition of the charging case 2. For example, a flashing green light indicates that recharging is in progress, a constant green light indicates that the recharging work has been completed; a flashing blue light indicates that ultraviolet disinfection work is in progress, and a constant blue light indicates that the disinfection work has been completed.

In this embodiment, the bottom holder 21 includes a first outer case 213 and a first inner case 214. The first outer case 213 and the first inner case 214 are plugged and fixed by a plurality of first limited columns 215. Passing through the second control mainboard 212, the limited columns are used to fix the second control mainboard 212 to prevent the second control mainboard 212 from moving inside the bottom holder 21. The upper cover 22 includes a second outer cover 222 and a second inner cover 223. The second outer cover 222 and the second inner cover 223 are plugged and fixed by a plurality of second limited columns 224. The second outer cover 222 and the second inner cover 223 clamp and fix the first magnet 221.

Further, in the case where the rehabilitation device provided by this embodiment is not needed, a second magnet 216 is arranged inside the bottom holder 21, and a third magnet 225 is arranged inside the upper cover 22 to ensure that the upper cover 22 is firmly covered on the bottom holder 21. In this embodiment, the second magnet 216 and the third magnet 225 have different magnetic properties and can be attracted to each other. In another embodiment, one of the second magnet 216 or the third magnet 225 has magnetic adsorption capability, and other is made of metal material, and can also be attracted to each other.

It could be understood that under the guidance of the above embodiments, those skilled in the filed can combine various implementation methods in the above embodiments to obtain technical solutions of multiple implementation methods.

The above description is only a preferred embodiment of the present invention and is not to limit the present invention. Any modifications, equivalent substitutions and improvements made within the spirit and principles of the present invention should be included in the protection scope of the present invention.

We claim:

1. A circuit module for pelvic floor muscle rehabilitation device, comprises a first controller, a wireless communication module, an electromyographic signal collection module, a discharge module and a battery, wherein the first controller is electrically connected to the electromyographic signal collection module, the wireless communication module and the discharge module; the battery is electrically connected to the first controller for supplying power; the electromyographic signal collection module is used to collect electromyographic signals of users and send the signals to the first controller; the discharge module is used to send electrical stimulation to users; the first controller is connected to a terminal device through the wireless communication module.

2. The circuit module for pelvic floor muscle rehabilitation device according to claim 1, wherein the circuit module further comprises a wireless charging receiving module, which is electrically connected to the battery for recharging.

3. The circuit module for pelvic floor muscle rehabilitation device according to claim 1, further comprises a voltage module, wherein the voltage module is electrically connected to the first controller and the battery, and is used to transform the battery voltage before supplying to the first controller.

4. The circuit module for pelvic floor muscle rehabilitation device according to claim 1, further comprises a high voltage relay module, wherein the electromyographic signal collection module is electrically connected to the first controller and the high voltage relay module; and the electromyographic signal collection module is used to collect electromyographic signals of users and then send the signals to the first controller; the discharge module is electrically connected to the first controller and the high voltage relay module, and is used to send electrical stimulation to users; the first controller is used to turn on or off the high voltage relay module; when the high voltage relay module turn on, the electromyographic signal collection module is connected to the first controller; when the high voltage relay module turn off, the discharge module is connected to the first controller.

5. The circuit module for pelvic floor muscle rehabilitation device according to claim 1, comprises a motor driving module, wherein the motor driving module is electrically connected to the first controller.

6. A pelvic floor muscle rehabilitation device comprising a circuit module according to any one of claims 1 to 5, comprises a rehabilitation device body, wherein the rehabilitation device body comprises a rehabilitation working area and a holding area connected to the rehabilitation working area; the surface of the rehabilitation working area is provided with electrode slices, which are used to send electrical stimulation to the users and collect electromyographic signals of users; the rehabilitation device body is provided with an inner cavity provided with a battery and a first control mainboard inside; the battery is electrically connected to the first control mainboard, and the circuit module is arranged on the first control mainboard.

7. The pelvic floor muscle rehabilitation device according to claim 6, wherein a connection board is arranged in the inner cavity; the first controller is arranged inside the holding area; the connection board is arranged inside the rehabilitation working area, and two ends of the connection board are respectively connected to the electrode slices and the first controller.

8. The pelvic floor muscle rehabilitation device according to claim 6, further comprises a charging case, wherein the charging case comprises a bottom holder and an upper cover for covering the bottom holder; the bottom holder is provided with a trough adapted to the rehabilitation device body; a second controller and a wireless charging sending module are arranged inside the bottom holder.

9. The pelvic floor muscle rehabilitation device according to claim 8, wherein an ultraviolet light driving module and an ultraviolet light module are arranged inside the bottom holder; the ultraviolet light driving module is electrically connected to the second controller and the ultraviolet light module; the ultraviolet light is arranged in the ultraviolet light module and embedded in the inner wall of the trough.

10. The pelvic floor muscle rehabilitation device according to claim 8, wherein a Hall detection module is arranged inside the bottom holder; the Hall detection module is electrically connected to the second controller, and a first magnet is arranged beneath the upper cover.
